(19) United States
(12) Patent Application Publication
(10) Pub. No.: US 2018/0201601 A1
(43) Pub. Date: Jul. 19, 2018

(54) PROCESS FOR PREPARING APALUTAMIDE

(71) Applicant: ScinoPharm Taiwan, Ltd., Shan-Hua (TW)

(72) Inventors: Shang-Hong Chen, Shan-Hua (TW); Jiunn-Cheh Guo, Shan-Hua (TW); Wen-Li Shih, Shan-Hua (TW)

(21) Appl. No.: 15/873,207

(22) Filed: Jan. 17, 2018

Related U.S. Application Data
(60) Provisional application No. 62/447,699, filed on Jan. 18, 2017.

(51) Int. Cl. C07D 401/04; C07C 237/30

(52) U.S. Cl. CPC C07D 401/04; C07C 237/30

(57) ABSTRACT

The present invention provides efficient, economical, and environmentally friendly processes for synthesizing apalutamide and intermediates thereof. Also provided herein are novel compounds and intermediates thereof.

### CROSS-REFERENCES TO RELATED APPLICATIONS

This application claims the benefit of priority to U.S. Provisional Application Ser. No. 62/447,699 filed Jan. 18, 2017, the entirety of which is incorporated herein by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Apalutamide (formerly known as ARN-509 or JNJ-56021927), which is chemically named as 4-[7-[6-Cyano-5-(trifluoromethyl)pyridin-3-yl]-8-oxo-6-sulfanylidene-5,7-diazaspiro[3.4]octan-5-yl]-2-fluoro-N-methylbenzamide, is a non-steroidal antiandrogen that is under development for the treatment of prostate cancer. It is similar to enzalutamide both structurally and pharmacologically, acting as a selective competitive antagonist of the androgen receptor (AR), but shows some advantages, including greater potency and reduced central nervous system permeation. Apalutamide is currently in phase III clinical trials for castration-resistant prostate cancer.

Publications related to apalutamide disclose several synthetic approaches. The synthetic approaches are described below.

In WO 2007/126765 and WO 2008/119015, pyridine carbonitrile 1 was reacted with thiophosgene led to compound 2 in 74-95% yield (FIG. 1). Apalutamide was prepared by reacting Isothiocyanate 2 with methyl benzamide 3 in microwave then hydrolysis. Apalutamide was purified in 35-87% yield after column purification (acetone/DCM (5/95)). The synthetic approach is very limited for industrial application because microwave was not easy to apply in large scale synthesis and results in higher costs.

A similar approach for apalutamide preparation was also reported in WO 2008/119015 (FIG. 2). In a one pot reaction, pyridine carbonitrile 1 and thiophosgene were reacted followed by further reacting the product with methyl benzamide 3 then hydrolysis to afford apalutamide in 64-76% yield after column purification (acetone/DCM (5/95)). However, a highly toxic reagent (NaCN) was used to prepare compound 3 in this approach.

Although approaches for preparing apalutamide have been disclosed as discussed above, there is still an unmet need for a more environmentally friendly, industrially practical, and economical process for preparation of apalutamide. The present processes disclosed herein address this need and other needs.

### BRIEF SUMMARY OF THE INVENTION

In one aspect, the present disclosure provides a process for the preparation of apalutamide

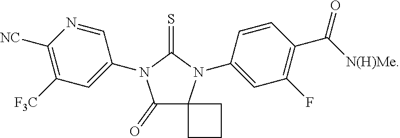

The process includes:
contacting a compound of Formula II

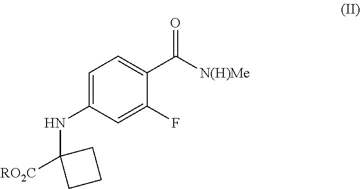

with a compound of Formula III

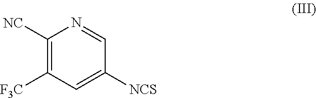

to provide apalutamide;
wherein R is C₁-C₆ alkyl.

In another aspect, the present disclosure provides a process for the preparation of apalutamide

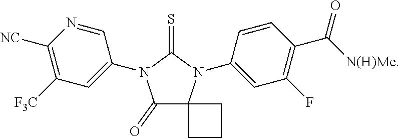

The process includes:
(a) contacting Starting Material 1 (SM1)

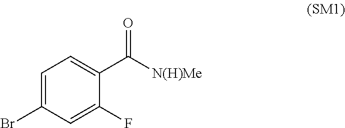

with Starting Material 2 (SM2)

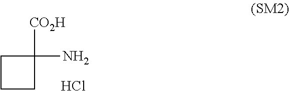

to provide a compound of Formula I

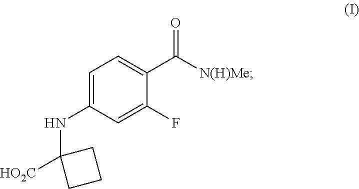

(b) contacting the compound of Formula I with an O-alkylating agent to provide a compound of Formula II

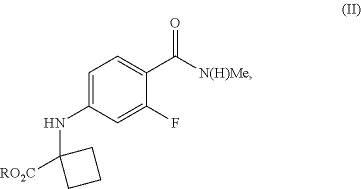

wherein R is C₁-C₆ alkyl; and
(c) contacting the compound of Formula II with a compound of Formula III

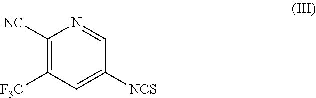

to provide apalutamide.

In another aspect, the present disclosure provides a compound of Formula II

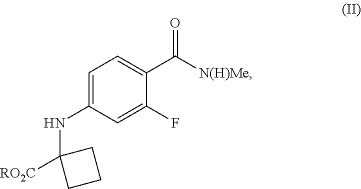

wherein R is C₁-C₆ alkyl.

### BRIEF DESCRIPTION OF THE DRAWINGS

FIG. 1 shows the synthetic route for apalutamide disclosed in WO 2007/126765 and WO 2008/119015.

FIG. 2 shows the synthetic route for apalutamide disclosed in WO 2008/119015 (one-pot reaction).

FIG. 3 shows the synthetic route for apalutamide described in the present application.

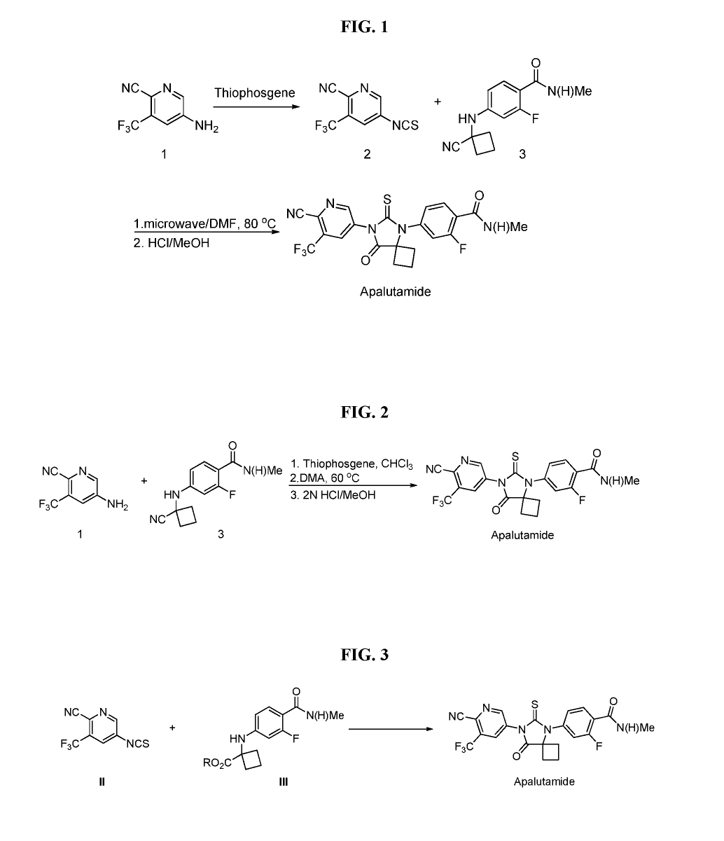

### DETAILED DESCRIPTION OF THE INVENTION

### I. General

The present invention provides improved processes for the preparation of apalutamide and intermediates thereof. The disclosed process is particularly advantageous because it avoids using highly toxic chemicals and chemical conversions that are difficult to control in larger scale syntheses. Both of these features make the discloses processes highly suitable for efficient and cost effective industrial scale synthesis.

While a complete synthetic scheme is provided in the detailed description, as well as the Examples, one of skill in the art will appreciate that selected steps of the instant process are novel and can be performed independent of the origin of the starting material or intermediates.

It will also be apparent to a person of skill in the art that some of the compounds and intermediates used in the disclosed process are novel.

### II. Definitions

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents which can be produced in the reaction mixture.

As used herein, the term “alkyl” by itself or as part of another substituent, means, unless otherwise stated, a straight or branched chain hydrocarbon radical. Alkyl substituents, as well as other hydrocarbon substituents, may contain number designators indicating the number of carbon atoms in the substituent (i.e. C₁-C₈ means one to eight carbons), although such designators may be omitted. Unless otherwise specified, the alkyl groups of the present invention contain 1 to 12 carbon atoms. For example, an alkyl group can contain 1-2, 1-3, 1-4, 1-5, 1-6, 1-7, 1-8, 1-9, 1-10, 1-11, 1-12, 2-3, 2-4, 2-5, 2-6, 3-4, 3-5, 3-6, 4-5, 4-6 or 5-6 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein, the term “one-pot reaction” refers to a reaction in which a starting material undergoes at least two sequential chemical transformations in a single reaction vessel. In general, compounds formed as intermediates in the sequence are not isolated from a one-pot reaction mixture. Reagents necessary to affect the transformation sequence may be added together at the beginning of the sequence, or they may be added one after another as the sequence progresses.

As used herein, the term “O-alkylating agent” refers to a chemical compound that causes the replacement of a hydrogen attached to an oxygen atom with an alkyl group. An O-alkylating agent is a chemical compound that provides the alkyl group in the reaction. O-alkylating agents may be used alone or in combination with a catalyst. In some embodiments, the catalyst used is a base. In some embodiments, O-alkylating agents catalyze the conversion of a carboxylic acid to an ester. The alkyl group provided by O-alkylating agents may be any suitable alkyl group. In particular embodiments, O-alkylating agents provide alkyl groups that are C₁-C₆ in length. In some embodiments, O-alkylating agents provide a C₁ alkyl. It is understood that alkenyl or alkyl groups may be used as O-alkylating agents without departing from the scope of the invention.

### III. Embodiments of the Invention

In one aspect, the present invention provides a process for the preparation of apalutamide:

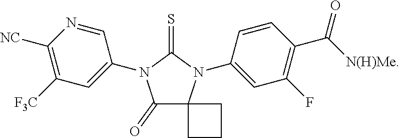

The process includes:
contacting a compound of Formula II

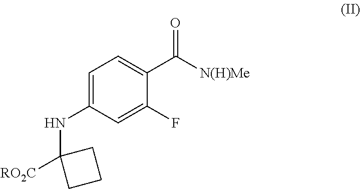

with a compound of Formula III

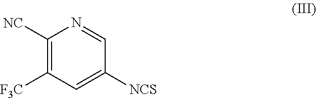

to provide apalutamide,
   wherein R is C₁-C₆ alkyl.

In some embodiments, the above process is conducted in an organic solvent selected from the group consisting of dimethylacetamide (DMAc), acetonitrile (MeCN), tetrahydrofuran (THF) and mixtures thereof. In some embodiments, the organic solvent is MeCN.

In some embodiments, over 1 equiv. of the compound of Formula III is used relative to the compound of Formula II in the above process. In some embodiments, 1.5-5.0 equiv. of the compound of Formula III is used relative to the compound of Formula II in the above process. It is understood that the equivalents of the compound of Formula III are relative to the compound of Formula II in the above process. In some embodiments, 2.0-5.0 equiv. of the compound of Formula III is used in the above process. In some embodiments, 3.0-5.0 equiv. of the compound of Formula III is used in the above process. In other embodiments, 3.0-4.5 equiv. of the compound of Formula III is used in the above process. In other embodiments, 3.5-4.5 equiv. of the compound of Formula III is used in the above process. In some embodiments, about 4.0 equiv. of the compound of Formula III is used in the above process. In other embodiments, about 4.5 equiv. of the compound of Formula III is used in the above process. In some embodiments, more than 5.0 equiv. of the compound of Formula III is used in the above process. When using 1.5 equiv. of the compound of Formula III in the cyclization step, the conversion yield of apalutamide obtained in the resulting mixture was 11-18%. For example, the conversion yield is 18% when using MeCN as the solvent; and the conversion yield is 11% when using DMAc as the solvent. When increasing the equivalent of the compound of Formula III from 1.5 to 3.9 equiv., and the resulting conversion yield of apalutamide was improved from 18 to 75%. When using 4.5 equiv. of the compound of Formula III, the conversion yield of apalutamide obtained in the resting the mixture was 80-88%. Apalutamide was isolated in 48-62% yield after workup followed by recrystallization from IPA.

In some embodiments, the reaction yield of apalutamide can be increased by the incremental addition of the compound of Formula III. In such embodiments, the total amount of the compound of Formula III is added in incremental steps allowing for the reaction to proceed after each individual addition. In some embodiments, the compound of Formula III is added in 2, 3, 4, 5, or more discrete increments during the reaction. In some embodiments, the compound of Formula III is added in 2 to 8 discrete increments during the reaction. In some embodiments, the compound of Formula III is added in 4 discrete increments during the reaction. In other embodiments, the compound of Formula III is added in 5 discrete increments during the reaction. In other embodiments, the compound of Formula III is added in 6 discrete increments during the reaction. It is understood that incremental addition may include adding different amounts of the compound of Formula III to the reaction. In some embodiments, 1.5 equiv. of the compound of Formula III is used in the initial reaction, and then each portion of 0.4 to 0.7 equiv. of the compound of Formula III is further added during different time points (e.g., 1-6 times) of the reaction. In some embodiments, 1.5 equiv. of the compound of Formula III is used in the initial reaction, and then each portion of 0.5 equiv. of the compound of Formula III is further added during different time points (e.g., 1-6 times) of the reaction. In other embodiments, 1.5 equiv. of the compound of Formula III is used in the initial reaction, and then each portion of 0.6 equiv. of the compound of Formula III is further added during different time points (e.g., 1-6 times) of the reaction.

In some embodiments, the above reaction is conducted at a temperature above 50° C. In some embodiments, the above reaction is conducted at a temperature above 60° C. In some embodiments, the above reaction is conducted at a temperature above 70° C. In some embodiments, the reaction temperature is from about 70-90° C. In some embodiments, the reaction temperature is from about 75-80° C. In some embodiments, the reaction temperature is from about 70-80° C. In some embodiments, the reaction temperature is from about 70-85° C. In other embodiments, the reaction temperature is from about 75-85° C. In other embodiments, the reaction temperature is from about 75-90° C.

In some embodiments, R is C₁-C₄ alkyl. Non-limiting examples of C₁-C₄ alkyl include methyl, ethyl, isopropyl, and n-butyl. In some embodiments, R is methyl.

In some embodiments, the process includes:
contacting a compound of Formula IIa

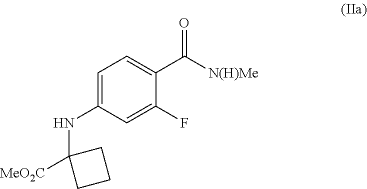

with a compound of Formula III

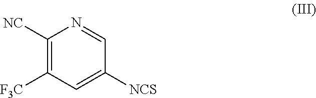

to provide apalutamide.

The above process is conducted in the organic solvent with over 1 equiv. of the compound of Formula III relative to the compound of Formula IIa at the temperature, as described herein.

In some selected embodiments, the process includes:
contacting a compound of Formula IIa

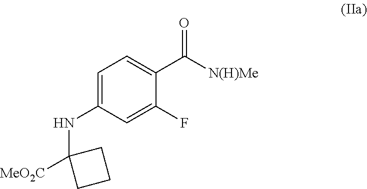

with a compound of Formula III

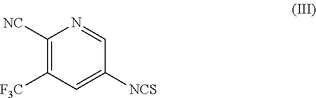

to provide apalutamide;
wherein 3.0-5.0 equiv. of the compound of Formula III is used relative to the compound of Formula IIa.

In some selected embodiments, about 4.0 equiv. of the compound of Formula III is used in the above process. In other selected embodiments, about 4.5 equiv. of the compound of Formula III is used in the above process.

The compound of Formula III can be added in 2, 3, 4, 5, or more discrete increments during the reaction, as described herein. In some selected embodiments, the compound of Formula III is added in 5 discrete increments during the reaction. In other selected embodiments, the compound of Formula III is added in 6 discrete increments during the reaction.

The incremental addition can include adding different amounts of the compound of Formula III to the reaction, as described herein. In some selected embodiments, 1.5 equiv. of the compound of Formula III is used in the initial reaction, and then each portion of 0.4 to 0.7 equiv. of the compound of Formula III is further added during different time points (e.g., 1-6 times) of the reaction.

The synthesis of apalutamide in the present invention is conducted without the use of highly toxic reagents (such as NaCN) and microwave conditions.

In some embodiments, the compound of Formula II is prepared by a process comprising:
contacting the compound of Formula I with an O-alkylating agent

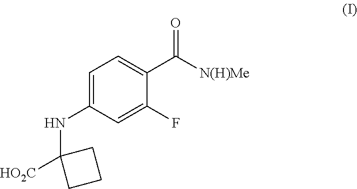

to provide a compound of Formula II

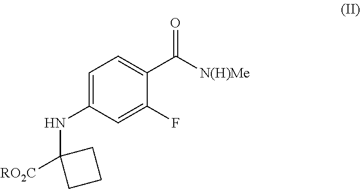

wherein R is C₁-C₆ alkyl.

The compound of formula II can be made using a variety of transformation conditions that are well known to a person of skill in the art. In some embodiments, the transformation is a transesterification reaction. In some embodiments, the transformation is an O-alkylation reaction.

In some embodiments, the O-alkylation is performed in the presence of a base. A number of bases are suitable for this conversion. Suitable bases include, but are not limited to, Li₂CO₃, K₂CO₃, Cs₂CO₃, Na₂CO₃, NaHCO₃, KHCO₃ or a combination thereof. In some embodiments, the base used is K₂CO₃.

In some embodiments, the O-alkylating agent is selected from the group consisting of RI, RBr, and RCl, wherein R is C₁-C₆ alkyl. In some embodiments the O-alkylating agent is RBr. In some embodiments the O-alkylating agent is RI. In some embodiments, R is C₁-C₄ alkyl. Non-limiting examples of C₁-C₄ alkyl include methyl, ethyl, isopropyl, and n-butyl. In some embodiments, R is C₁ (methyl). In some embodiments, the O-alkylating agent is selected from the group consisting of CH₃I, CH₃Br, and CH₃Cl. In some embodiments, the O-alkylating agent is CH₃Br. In other embodiments, the O-alkylating agent is CH₃I.

In some embodiments, the O-alkylation is performed in a polar solvent. In some embodiments the polar solvent is selected from the group consisting of dimethylacetamide (DMAc), dimethylformamide (DMF), dimethyl sulfoxide (DMSO), 1-methyl-2-pyrrolidone (NMP), isopropyl acetate (IPAc), and mixtures thereof. In some embodiments, H₂O is mixed with the polar solvent in the O-alkylation. Preferably, small or catalytic amount of H₂O is mixed with the polar solvent. In those embodiments, the O-alkylation is performed in a mixture of a polar solvent and H₂O. In some embodiments, the mixture comprises DMAc and H₂O.

In some selected embodiments, the O-alkylation is performed with the O-alkylating agent selected from the group consisting of CH₃I, CH₃Br, and CH₃Cl in the presence of the base in a mixture of the polar solvent and H₂O. The polar solvent and the base are as described herein. In some embodiments, the polar solvent is DMAc. In some embodiments, the base is K₂CO₃. In some selected embodiments, the O-alkylation is performed with the O-alkylating agent selected from the group consisting of CH₃I, CH₃Br, and CH₃Cl in the presence of K₂CO₃ in a mixture of DMAc and H₂O.

It is understood that the O-alkylation reaction show above may be performed at a variety of temperatures. In general, warming the reaction above room temperature increases the rate of the reaction. In some embodiments, the reaction is warmed to above 35° C. In some embodiments, the reaction is warmed to about 35-55° C., or 40-45° C.

In some embodiments, the O-alkylation yield is greater than 80 or 85%.

In another aspect, the present disclosure provides a process for the preparation of apalutamide

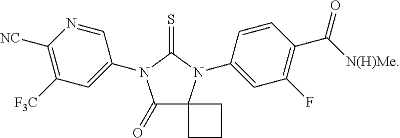

The process includes:
(a) contacting Starting Material 1 (SM1)

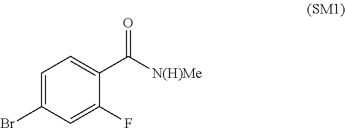

with Starting Material 2 (SM2)

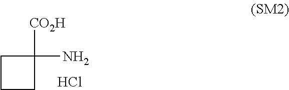

to provide a compound of Formula I

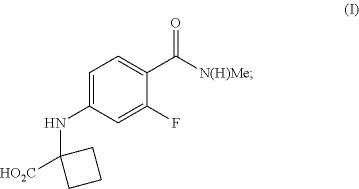

(b) contacting the compound of Formula I with an O-alkylating agent to provide a compound of Formula II

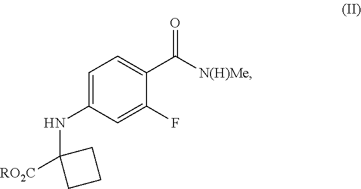

wherein R is C₁-C₆ alkyl; and
(c) contacting the compound of Formula II with a compound of Formula III

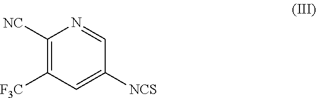

to provide apalutamide.

In some embodiments, the compound of Formula III is prepared by
(c-1) contacting Starting Material 3 (SM3)

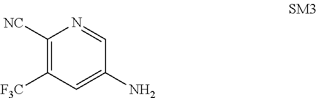 [SMILES: N#Cc1ncc(N)cc1C(F)(F)F]

with thiophosgene to provide the compound of Formula III, wherein step (c-1) is performed before adding the compound of Formula III to step (c).

The step (c-1) may be performed as described in WO 2007/126765, for example in water or in a biphasic mixture of chloroform and water. Preferably, the step (c-1) is performed in the same organic solvent used for step (c) wherein the organic solvent is selected from the group consisting of dimethylacetamide (DMAc), acetonitrile (MeCN), tetrahydrofuran (THF) and mixtures thereof. In some embodiments, the step (c-1) is performed in MeCN. In some embodiments, the compound of Formula III is used in step (c) without further purification.

In some embodiments, the conversion described in step (a) includes a base. A number of bases are suitable for this conversion. Suitable bases include, but are not limited to, Li₂CO₃, K₂CO₃, Cs₂CO₃, Na₂CO₃, NaHCO₃, KHCO₃ or a combination thereof. In some embodiments, the base used is K₂CO₃.

In some embodiments, the conversion described in step (a) includes a metal catalyst. In some embodiments, the metal catalyst is a copper salt. In some embodiments the copper salt is selected from the group consisting of CuCl, CuI, and mixtures thereof.

A person of skill in the art will recognize that a number of solvents are useful in the conversion of step (a). In some embodiments the solvent is selected from the group consisting of 2-acetylcyclohexanone/DMAc, 2,4-pentanedione, 2,4-hexanedione, 1-phenyl-1,3-butanedione/DMAc, DMF, DMSO, NMP and mixtures thereof.

In some embodiments, the process for the preparation of apalutamide includes
(a) contacting Starting Material 1 (SM1)

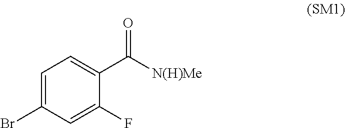

with Starting Material 2 (SM2)

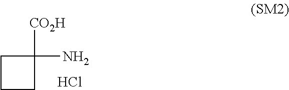

in the presence of a metal catalyst to provide a compound of Formula I

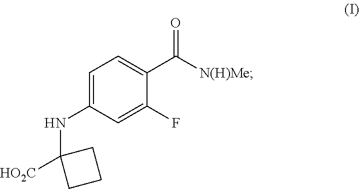

(b) contacting the compound of Formula I with an O-alkylating agent to provide a compound of Formula II

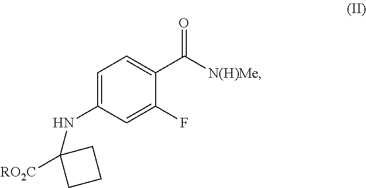

wherein R is C₁-C₆ alkyl; and
(c) contacting the compound of Formula II with a compound of Formula III

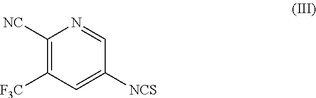

to provide apalutamide.

In some embodiments, the metal catalyst is a copper salt. In some embodiments, the copper salt is selected from the group consisting of CuCl, CuI, and mixtures thereof.

The conversion step (a) includes a base and a solvent, as described herein. In some selected embodiments, the base is K₂CO₃. In some selected embodiments, the solvent is 2-acetylcyclohexanone/DMAc.

The O-alkylation step (b) is as described herein. In some selected embodiments, the O-alkylating agent is CH₃I. In one selected embodiment, the O-alkylation is performed with CH₃I in the presence of K₂CO₃ in a mixture of DMAc and H₂O.

The conversion step (c) to apalutamide is as described herein. In some embodiments, 3.0-5.0 equiv. of the compound of Formula III is used. In some embodiments, the compound of Formula III is added in 2 to 8 discrete increments during the reaction. In some selected embodiments, 1.5 equiv. of the compound of Formula III is used in the initial reaction, and then each portion of 0.4 to 0.7 equiv. of the compound of Formula III is further added during different time points (e.g., 1-6 times) of the reaction.

In some selected embodiments, the compound of Formula II wherein R is methyl is a compound of Formula IIa.

In still another aspect, the present disclosure provides a compound of Formula II

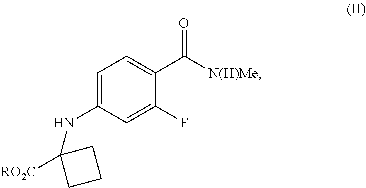

wherein R is C₁-C₆ alkyl.

In some selected embodiments, R is methyl and the compound of Formula II is a compound of Formula IIa:

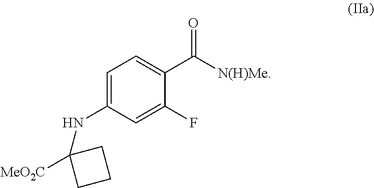

### IV. Examples

The following examples are presented to describe the invention in further detail. However, the present invention is by no means restricted to the specific embodiments described herein.

Abbreviations used are those commonly used in the art. Examplary abbreviations used include mL (milliliters), mmole (millimoles), equiv. (equivalents), DCM (dichloromethane), DMAc (dimethylacetamide), HOAc (acetic acid), IPAc (isopropyl acetate), MeCN (acetonitrile), IPA (isopropyl alcohol), min (minutes), vol. (volume), hr (hour), NLT (not longer than).

### Example 1: Synthesis of Compound of Formula I

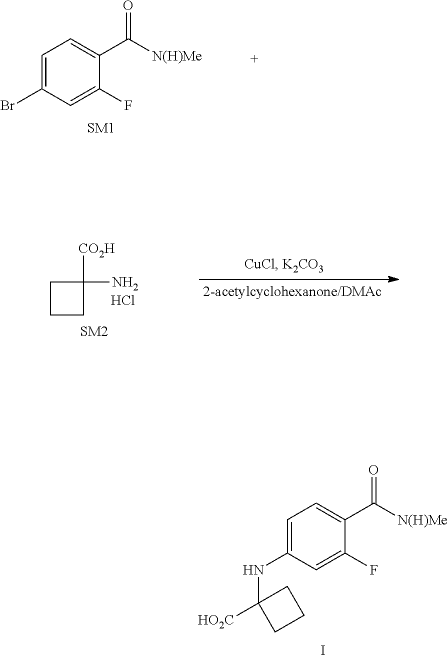

To a four-necked round bottom flask was equipped with a mechanical stirrer and a thermometer. To the flask was added SM1 (20 g), SM2 (19.6 g), K₂CO₃ (35.7 g), CuCl (1.7 g), 2-acetylcyclohexanone (2.42 g), DMAc (120 mL, 6 vol.) and H₂O (3.6 mL) at 20-30° C. under nitrogen. The mixture was heated to 95-105° C. and stirred for NLT 8 hr. H₂O (180 mL, 9 vol.) and DCM (240 mL, 12 vol.) were added into the mixture for quench and extraction. 2 N HCl_{(aq)} was added into the aqueous portion to adjust pH till 2-3. The mixture was filtered and the filter cake was washed with H₂O. The compound of Formula I was obtained as tan solids (19.45 g, 85% yield, 99.66% purity).

### Example 2: Synthesis of Compound of Formula IIa

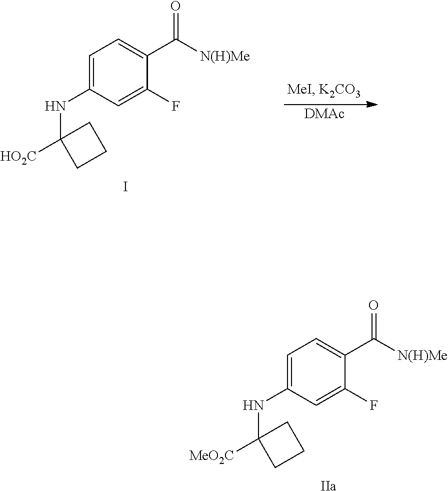

The compound of Formula I (18 g), K₂CO₃ (14 g), DMAc (126 mL) and H₂O (0.18 mL) were added into a four-necked round bottom flask equipped with a mechanical stirrer and a thermometer at 20-30° C. under nitrogen. The reaction mixture was warmed to 25-35° C. followed by adding MeI (11.5 g, 81.02 mmole, 1.2 equiv.). The reaction mixture was further warmed to 40-45° C. under nitrogen and stirred for NLT 1 hr. After the reaction was complete, HOAc (1.35 mL, 0.075 vol.) was added at 40-45° C. then warmed to 60° C. H₂O (270 mL, 15 vol.) was added slowly followed by cooling to 20-30° C. The mixture was filtered followed by washing with H₂O (36 mL, 2 vol.). The crude product was charged H₂O (180 mL, 10 vol.) and stirred for NLT 0.5 hr at 20-30° C. The mixture was filtrated and the filtrate cake was washed with IPAc (36 mL, 2 vol.) to obtain the compound of Formula IIa (16.82 g) in 89% yield with 99.86% purity.

### Example 3: Synthesis of Compound of Formula III

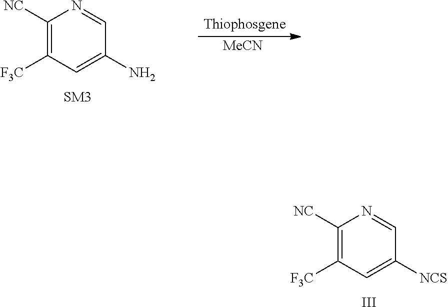

SM3 (20 g) and MeCN (300 mL) were added into a four-necked round bottom flask equipped with a mechanical stirrer and a thermometer at 20-30° C. under nitrogen followed by cooling to 0-10° C. Thiophosgene (13.7 mL) was added and the mixture was warmed to 20-30° C. After the reaction was completed, the reaction mixture was cooled to 0-10° C. and added saturated NaHCO₃ to adjust the pH value to 6-7. The organic layer was separated and concentrated to dryness. The compound of Formula III was obtained (24.93 g) in 100% yield as the brown oil.

### Example 4: Synthesis of Apalutamide

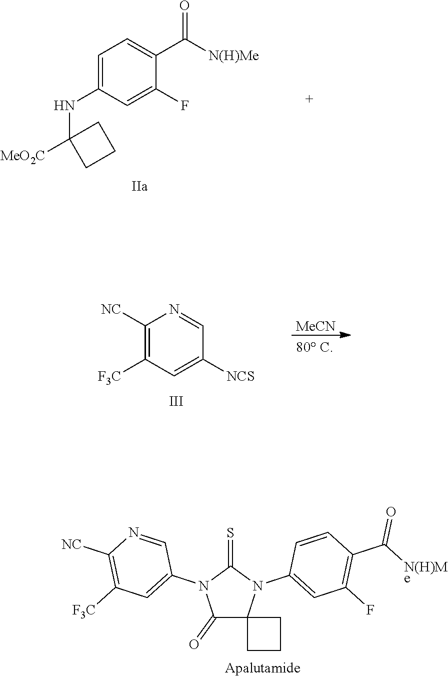

The compound of Formula IIa (5 g), MeCN (75 mL) and the compound of Formula III (6.14 g) were added into a four-necked round bottom flask equipped with a mechanical stirrer and a thermometer at 20-30° C. under nitrogen followed by warming to 75-85° C. and stirred for NLT 8 hr. After 8 hours, an additional aliquot of the compound of Formula III (2.45 g) was added to the reaction mixture at 75-85° C. and stirred for NLT 8 hr. This step was repeated 4 additional times (5 total aliquots of Formula III were added). After the reaction was completed, the reaction mixture was cooling to 0-10° C. and stir for 1 hr. The reaction mixture was filtered and the filtrate was concentrated to obtain crude apalutamide as the brown oil (191.88 g). The crude apalutamide was purified by using fresh column chromatography and hot slurry with IPA. Purify apalutamide was obtained as an off-white solid in 53.5% yield with 99.17% purity.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A process for the preparation of apalutamide 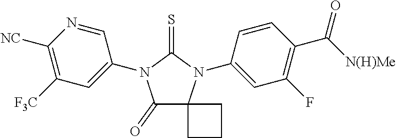
the process comprising:
contacting a compound of Formula II 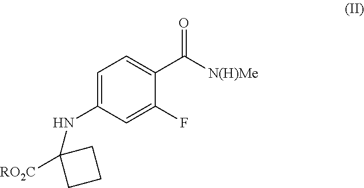
with a compound of Formula III 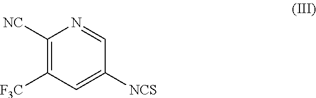
to provide apalutamide;
wherein R is C₁-C₆ alkyl.

2. The process of claim 1, wherein the reaction is conducted at a temperature above 50° C.

3. The process of claim 2, wherein the reaction is conducted at a temperature above 70° C.

4. The process of claim 2, wherein the reaction is conducted at a temperature from about 75-85° C.

5. The process of claim 1, wherein 1.5-5 equiv. of the compound of Formula III is used relative to the compound of Formula II.

6. The process of claim 5, wherein 4.5 equiv. of the compound of Formula III is used.

7. The process of claim 5, wherein the compound of Formula III is added incrementally.

8. The process of claim 1, wherein the reaction is conducted in an organic solvent.

9. The process of claim 8, wherein the organic solvent is selected from the group consisting of dimethylacetamide (DMAc), acetonitrile (MeCN), tetrahydrofuran (THF) and mixtures thereof.

10. The process of claim 9, wherein the organic solvent is acetonitrile.

11. The process of claim 1, wherein the compound of Formula II is prepared from a process comprising:
contacting the compound of Formula I with an O-alkylating agent 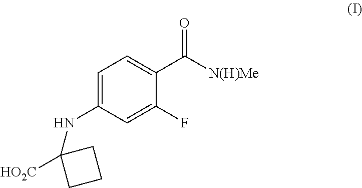
to provide a compound of Formula II,
wherein R is C₁-C₆ alkyl.

12. The process of claim 11, further comprising a base selected from the group consisting of Li₂CO₃, K₂CO₃, Cs₂CO₃, Na₂CO₃ and combinations thereof.

13. (canceled)

14. The process of claim 12, wherein the base is K₂CO₃.

15. The process of claim 11, wherein O-alkylating agent is selected from the group consisting of RI, RBr, and RCl, wherein R is C₁-C₆ alkyl.

16. The process of claim 15, wherein the O-alkylating agent is RI.

17. The process of claim 1, wherein R is methyl.

18. A process for the preparation of apalutamide 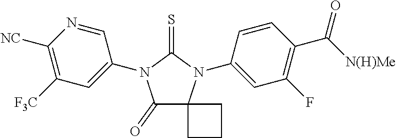
the process comprising:
(a) contacting Starting Material 1 (SM1) 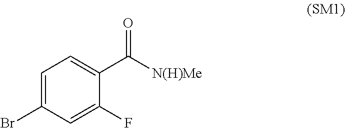
with Starting Material 2 (SM2) 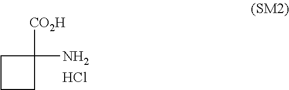
to provide a compound of Formula I 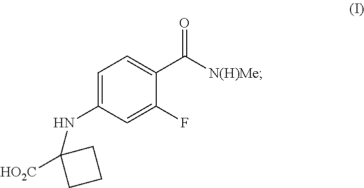
(b) contacting the compound of Formula I with an O-alkylating agent to provide a compound of Formula II 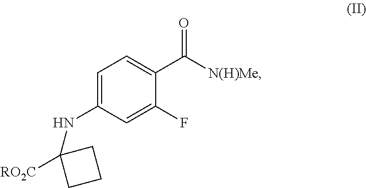
wherein R is C₁-C₆ alkyl; and
(c) contacting the compound of Formula II with a compound of Formula III 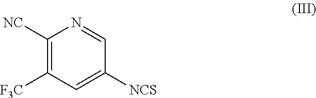
to provide apalutamide.

19. The process of claim 18, wherein step (a) is conducted in the presence of a metal catalyst.

20. The process of claim 19, wherein the metal catalyst is a copper salt selected from the group consisting of CuCl, CuI, and mixtures thereof.

21. A compound of Formula II 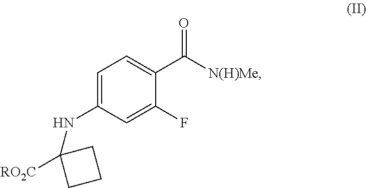
wherein R is C₁-C₆ alkyl.
